(19) United States
(12) Patent Application Publication
(10) Pub. No.: US 2019/0103177 A1
(43) Pub. Date: Apr. 4, 2019

(54) MEDICAL PERSONAL DATA CARD AND SYSTEM

(71) Applicant: F. Maury Matthews, Brentwood, MO (US)

(72) Inventor: F. Maury Matthews, Brentwood, MO (US)

(21) Appl. No.: 16/205,374

(22) Filed: Nov. 30, 2018

Related U.S. Application Data
(63) Continuation-in-part of application No. 15/867,076, filed on Jan. 10, 2018, now abandoned.
(60) Provisional application No. 62/444,447, filed on Jan. 10, 2017.

(51) Int. Cl. G16H 10/65; G06F 21/60

(52) U.S. Cl. CPC G16H 10/65; G06F 21/602

(57) ABSTRACT

A medical personal data card system utilizes a medical data card (MDC) containing a person's medical information which is stored in and retrieved from the cloud and stored in a data storage chip (DS) on the card. The information is encoded information, stored in an encrypted format, in compliance with HIPAA regulations. The card and the information stored on it are accessed and used by medical personnel under appropriate circumstances and with appropriate authorization. The card and the associated storage chip are the “key” “unlocking” access to the patient's discrete cloud account where all medical treatments are kept current without undue intervention of the patient's primary physician.

### REFERENCE TO RELATED APPLICATIONS

This application is a continuation-in-part application which is based upon and claims the benefit of U.S. non-provisional application Ser. No. 15/867,076 filed Jan. 1, 2018, which claims benefit from U.S. provisional patent application 62/444,447 filed on Jan. 10, 2017.

### BACKGROUND OF THE INVENTION

The need for, and the process of, digitizing individual medical records has been recognized for over 20 years and has been established as either EMR (Electronic Medical Records) or EHR (Electronic Health Records). EMRs are digitized versions of paperwork in a clinician's office and contain the medical and treatment history of patients at the clinician's practice. EHRs provide more information than EMRs as they focus on a patient's total health; not just standard clinical data, but a broader view of the health care being provided to the patient. EHRs facilitate sharing data outside a particular practice with other health care providers such as laboratories and specialists. Therefore, EHRs record information from all the clinicians involved in the patient's care.

As a part of the American Recovery and Reinvestment Act, all public and private healthcare providers and other eligible professionals (EP) were required to adopt and demonstrate “meaningful use” of EMRs by Jan. 1, 2014 in order to maintain their existing Medicaid and Medicare reimbursement levels. Since that date, the use of electronic medical and health records has spread worldwide and demonstrated its many benefits to health organizations everywhere. The industry that evolved to design, transfer, and sell medical facilities the wherewithal to accomplish this mandated transition has, unfortunately, not fully developed a system that is transparent and easily shared with medical facilities utilizing differing digital formats.

The result is that there is an impediment to effective and timely transmission of both EMR and EHR data between institutions in a patient emergency. This critical need has driven the necessity for a device; i.e., a personal Medical Data Card (“MDC”) that is able to immediately provide detailed medical information in an emergency condition (EMT) or in a hospital where such medical history and diagnostic data can shortcut unnecessary medical testing (CATSCANS, MRIs or other invasive procedures). Since the MDC also provides the information of the patient's primary physician, actions otherwise taken in a vacuum now have a resource(s) to provide guidance for treatment.

The present invention relates to a person's medical information; and, more particularly, to a medical personal data card system containing the person's medical information, which can be carried by the person, which can be accessed and used by authorized medical personnel under appropriate circumstances, and which complies with the Health Insurance Portability and Accountability Act or HIPAA.

As is well-known in the medical field, HIPAA includes privacy provisions that prevent protected patient information from being communicated from one party, person, or entity, to another party, person, or entity unless certain guidelines are followed.

While patient confidentiality is important, the regulations imposed under HIPAA in some circumstances act as a hindrance when there is a need for medical personnel to critically access vital patient information. Such situations include, for example, when an emergency responder at an accident scene needs information to treat a victim while insuring that the person will not have a negative reaction to an injection of some medication.

An “authorized” individual accessing the medical history of the patient must enter an authorizing code (i.e., an algorithm) containing information such as today's date and time, a vehicle (i.e., ambulance) or facility (e.g., hospital) code and the user's personal ID number along with a biometric of the user such as a fingerprint. The resulting discrete code and biometric (collectively, the “Personal ID”) is maintained by the hospital or other authorizing agency whether an EMT, fire department, hospital or other certified agency dealing with emergences and or physical/medial crisis as determined by the state, county or municipality. The Personal ID determines the individual's authority to access the patient's MDC and the level of access allowed.

The circumstances involving use of a person's MDC include medical emergencies, particularly when the person is undergoing emergency treatment by an EMT, or other qualified and trained person, or the person is being treated at a location distant from their home. However, in more mundane settings, the need to readily access vital information may also arise. For example, a person is visiting their doctor but, for some reason, the doctor's internal system breaks down and prevents the doctor or his or her staff from accessing the person's medical information. While not necessarily an emergency, it is certainly an inconvenience to both the person and the doctor if necessary information cannot be readily accessed.

The MDC is usually initiated by the patient's primary physician and they act as the patient's referring conduit to other medical specialists or institutions. Should the patient not have a primary physician, the clinic, hospital, or other medical authorities shall become the initiator of the MDC.

### SUMMARY OF THE INVENTION

The present disclosure is directed to a medical data card or MDC and an associated system for its use in a variety of circumstances.

The card is, for example, a plastic (PVC type) card with a read/write chip which is carried by someone on their person (e.g., on a bracelet or necklace, lanyard or on their body) and contains the person's medical information stored in compliance with HIPAA regulations.

The purpose of the MDC and the devices associated with it are to allow access to personal patient information, complying HIPAA regulations, during emergency situations when the individual of the card is, for example, either away from his/her home due to travel, or is in transit to an emergency room at a local hospital. The need for accessibility is to allow potential life saving treatment during transit to and during hospital treatment at an emergency room. The MDC also provides contact information for the patient including their family and medical professionals who ordinarily treat them, as well as information commonly included in a Living Will. The card is readily and affordable and the card and the associated devices allow access only to authorized medical personnel.

The system utilizes the “cloud” for information storage and retrieval and the devices medical data and personal medical information to a chip, a reader, and an optical scanner, etc. The card includes a segment where optical and scanable secure information is stored.

The data storage chip has the capacity to hold a gigabyte+/− of text, data, and/or images which are scanned, read and/or saved to a medical record.

The chip also lists all of the person's active and recent prescriptions drugs, as well as allergic or adverse reactions to them, and their dosage, as well as ongoing medical treatments for diseases such as cancer (i.e., chemotherapy and/or radiation) and/or other medical treatments.

The card and devices are respectively affordable by the individual and by medical institutions needing to access the MDCs

Other objects and features will be in part apparent and in part pointed out hereinafter.

### BRIEF DESCRIPTION OF THE DRAWING

FIG. 1. Is a representation of a medical data card of the present invention; and,

FIG. 2 illustrates use of a system to access medical data stored on the card.

### DETAILED DESCRIPTION OF INVENTION

The following detailed description illustrates the invention by way of example and not by way of limitation. This description clearly enables one skilled in the art to make and use the invention, and describes several embodiments, adaptations, variations, alternatives and uses of the invention, including what is presently believed to be the best mode of carrying out the invention. Additionally, it is to be understood that the invention is not limited in its application to the details of construction and the arrangement of components set forth in the following description or illustrated in the drawings. The invention is capable of other embodiments and of being practiced or carried out in various ways. Also, it will be understood that the phraseology and terminology used herein is for the purpose of description and should not be regarded as limiting.

As previously noted, while patient confidentiality is important, the regulations imposed under HIPAA in some circumstances act as a hindrance when there is a need for medical personnel to critically access vital patient information.

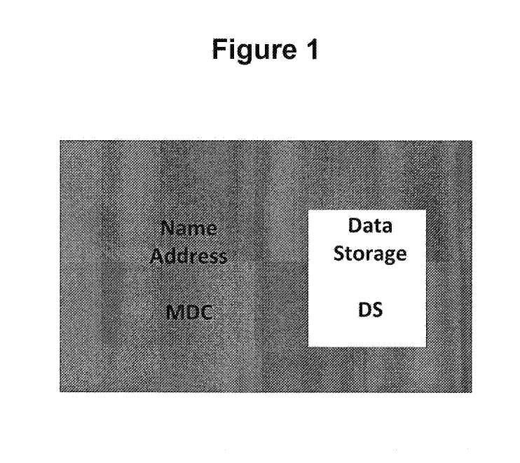

As shown in FIG. 1, a person's medical data card MDC card is, for example, a molded plastic card similar, for example, to a conventional credit card in size and shape. The MDC includes the person's name and current address, date of birth, as well as other pertinent information, if appropriate. The card also includes an optical data storage chip DS for storing all of the person's medical records. It will be understood by those skilled in the art that the MDC is a vehicle for the person to carry chip DS and the medical information stored on it. The data chip with its patient information can also be carried on the person by other means, as for example, a bracelet worn on the wrist, or a necklace worn around the neck.

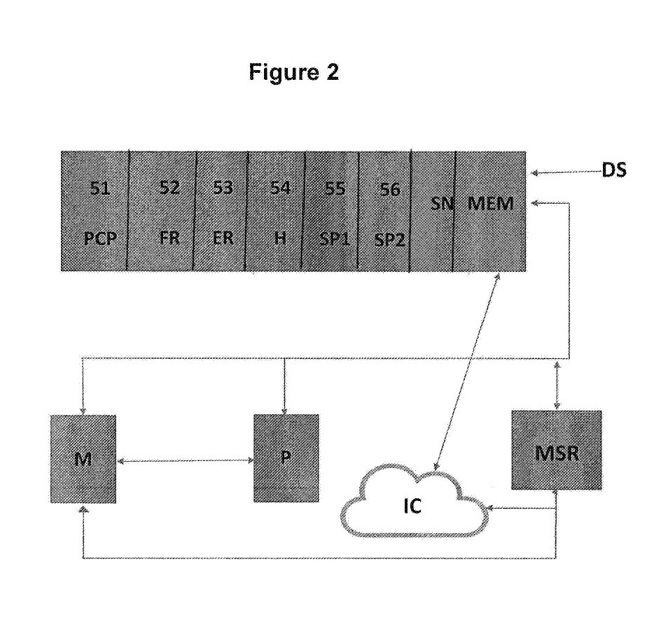

As shown in FIG. 2, the MDC is created by connecting the chip portion of the card to a medical IT system IT that holds the patient's medical records containing, for example, pertinent medical tests, x-rays, MRIs etc. These records are written to the data storage chip in “plain English” but are stored on the chip in an encoded, encrypted format. For this purpose, chip DS has a memory MEM storage capacity on the order of at least 1 gigabyte.

Information stored in chip DS can be read by a magnetic stripe reader MSR, see FIG. 2. Such readers read/decode the optical storage utilizing a series of lens that transforms the stored data into a format that can be read on a linked monitor M by an authorized medical professional, copied to a data file in, for example, the internet cloud IC, or linked to a printer P.

As shown in FIG. 2, data storage chip DS is partitioned into a series of segments S1-Sn with an authorized user only being able to access information pertinent to an activity undertaken by that user. The person's primary care physician PCP can, for example, access segment 1. Specialists SP treating the person for particular illnesses can access segments SP1, SP2, etc.; although, each specialist can only access that partitioned segment related to the illness for which he/she is treating the patient. It will be understood by those skilled in the art that much person/patient information (name, date of birth, basic medical history, may well be available through accessing any of the segments. Nonetheless, in compliance with HIPAA regulations, this information is not commonly available, but is separately accessed in each segment. Information unique to a person's particular medical condition may only be uniquely available in the data chip DS segment related to that condition. It will be understood by those in the art that not only is a person's medical information stored in the cloud stored separately and uniquely from that of other persons, but also that the person's medical information is stored such that information relevant to one partitioned segment on card DS is only available to a person properly accessing that segment.

In this regard, a first responder FR, for example, may only be able to access information in segment S2 of the chip. This information may include, for example, the person's, name, age and date of birth, medical conditions (angina, diabetes, hyper-tension, emphysema, coronary heart disease and other conditions) the knowledge of which will assist the responder in taking appropriate medical measures. Importantly, data storage chip DS includes a GPS function that, when activated, provides accurate location and time of the first responder's initial contact with the person.

If the person is subsequently treated in an emergency room ER, ER personnel can only access segments S2 and S3 of the chip. It will be understood by those skilled in the art that if the person is subsequently admitted to the hospital H, hospital H staff can access segments S2, S3, and S4. Again, each partitioned segment is only accessible by the appropriate personnel associated with the particular segment. At each separate step in the above exemplary sequence, access to the patient's cloud IC account and medical history must again require a facility code (Entry), and an individual's authorized identification code including a biometric verification (Access). This establishes not only the proper and necessary authorization, but also, the location, name of the facility (e.g., emergency room, hospital), and the time. Further, when access is granted in the ER, the patient's medical history is downloaded to the hospital's medical/patient's Electronic Medical Record (EMR).

Per today's standard operating procedures in hospitals and other treatment facilities, access to medical records now shifts to, for example, a patient wrist identification unit that the patient is given to wear and which is used to track all medical procedures taking place at the facility. In such instances circumstances, if the person does not have their MDC, a medical alert (MEDALERT) provider can provide this to them to wear.

The MDC is quite literally the key to any given patents' medical record(s) utilizing multiple layers of encrypted user identification as the “gatekeeper” to authorized access and in full compliance with HIPAA.

Of utmost importance, and the first requirement of the MDC and the discrete cloud account, is absolute patient confidentiality conforming to HIPAA regulations. Security is achieved when the chip on the MDC, or other carrier, is scanned by the card (chip) reader triggering an “entry” code requirement that identifies the vehicle utilizing an alphanumeric portion of the vehicle's vin number and the vehicle's number assigned by the jurisdiction and/or company; an “access” code comprised of the individual's unique identification code that must be entered correctly within a finite time and iteration constraint; or a “biometric” verifying the match of the “access” code to the authorized user. Numerous states (>25%) now require fingerprinting of all medical professionals including EMT/EMS allowing for a biometric security test allowing for a medical professional access to patient information and providing a tracking of each and every MDC access by name, date, job description, and locale. If a biometric is unavailable due to a state's not requiring fingerprinting of all medical professionals; an alternative security check will employ a digital photo of the “authorized” user against a stored employee record of the “authorized” user. This would also apply to a medical professional not involved with a medical crisis or hospitalization but requiring a patient's medical history in a “remote” location.

The MDC and the “cloud” account established by the continuation in part application, must be partitioned precluding access to information not necessary for the level of care provided. In other words, and EMT/First Responder has access only to medical history/data required to perform life saving measures (partition one) in the first 20 minutes of transporting a patient to an emergency room. The ER staff is allowed access to partition one and two as more inclusive information is required for proper medical care until the patient is either admitted to the hospital or discharged. After admission to the hospital pertinent medical personal have access to all of the partitions one, two, and three.

This provides not only patient security it also restricts access to information not necessary to the level of care provided.

The MDC is the portal to the cloud IC account. The MDC chip DS as the base of the person's medical information and all other pertinent information such as contacts (both family and doctors), durable medical powers, prescriptions, allergies, etc. Previously, there was a choke point created because the person's primary care physician PCP was the control point for medical information which had to be physically rewritten to chip DS each and every time there was an update. By utilizing chip DS as a portal to the cloud IC, any new information can be uploaded to the patient's cloud account automatically eliminating the choke point.

The MDC is readily available to the medical professionals involved in the administration of the person's medical treatment. The card with its partitioned data storage chip DS is readily carried by the person (e.g., in their wallet) and easily identified as a MDC. Data storage chip DS is easily read by a card reader (similar to a credit card) once a series of security codes are entered and a scan of a biometric (fingerprint) qualifies a medical personnel as someone to whom access to the medical data can be granted. Chip DS is read/write capable so records of treatment are archived and partitioned in accordance with HIPAA. Updating the person's cloud account is easily accomplished and readily automated.

Importantly, every time the cloud account is accessed, an email notification is sent to the primary care physician PCP of the access, the time of the access, where, and by whom. The treatment rendered and any other pertinent information to be included into the patient's permanent medical file is also indicated. This notification also triggers a billing event related for each access, as appropriate for insurance and related purposes.

The cloud account is unique to each separate person and is accessible only by the primary care physician PCP to make updates or changes in the medical data stored; unless the patient and their PCP agree to open access to the account to another medical professional because of a change in the person's residency or a change in the primary care physician.

Access to a patient's MDC is at least nationwide and is separate from existing EHRs so to allow universal accessibility to a patient's medical data/history given the appropriate security clearance. The capability for worldwide accessibility is being planned.

It will be understood that, pursuant to the procedure or test performed on the person, or to which the person is subjected, the information on the card can be updated. This may occur after each procedure or test, or the completion of all procedures and tests.

In view of the above, it will be seen that the several objects and advantages of the present disclosure have been achieved and other advantageous results have been obtained.

Having thus described the invention, what is claimed and desired to be secured by Letters Patent is:

1. A medical personal data system compliant with HIPAA regulations comprising:
a data storage means containing a person's medical information;
a vehicle on which the data storage means is carried, the vehicle being worn by or carried on the person;
means for accessing an information storage to uniquely store a person's medical information in the cloud and transfer the medical information back and forth between the cloud and the data storage means; and
wherein the data storage means is partitioned into a plurality of segments each of which includes a person's medical information related only to a specific medical condition or situation, and each segment being accessible only to those medical or medically related personnel authorized to view the information associated with that segment.
and a device for accessing the information stored on the card, the information being encoded information stored in an encrypted format in compliance with HIPAA regulations, the card and the information stored on it being accessed and used by medical personnel under appropriate circumstances.

2. The system of claim 1 in which the data storage means comprises a data storage chip carried by or on the vehicle.

3. The system of claim 2 in which the vehicle is a medical data card.

4. The system of claim 1 in which the information stored on the data storage means is stored in and retrieved from the cloud.

5. The system of claim 1 wherein the information stored in the cloud and on the card is encoded information stored in an encrypted format in compliance with HIPAA regulations, the card and the information stored on it being accessed and used by medical personnel only under appropriate circumstances.

6. A medical personal data system compliant with HIPAA regulations comprising:
a data storage chip containing a person's medical information, the data storage means being partitioned into a plurality of segments each of which includes a person's medical information related only to a specific medical condition or situation, and each segment being accessible only to those medical or medically related personnel authorized to view the information associated with that segment;
a medical data card on which the data storage means is contained, the card being carried by the person;
means for accessing an information storage cloud to uniquely store the person's medical information in the cloud and transfer the medical information back and forth between the cloud and the data storage means; and
wherein the information stored in the cloud and on the card is encoded information stored in an encrypted format in compliance with HIPAA regulations, the card and the information stored on it being accessed and used by medical personnel only under appropriate circumstances.
